(19) United States
(12) Patent Application Publication
(10) Pub. No.: US 2018/0042595 A1
(43) Pub. Date: Feb. 15, 2018

(54) VALVE RAKE AND MOUNT FOR SURGICAL RETRACTOR

(71) Applicant: TERUMO CARDIOVASCULAR SYSTEMS CORPORATION, ANN ARBOR, MI (US)

(72) Inventor: TAKESHI TSUBOUCHI, DEXTER, MI (US)

(21) Appl. No.: 15/669,388

(22) Filed: Aug. 4, 2017

Related U.S. Application Data
(60) Provisional application No. 62/372,334, filed on Aug. 9, 2016.

(51) Int. Cl. A61B 17/02

(52) U.S. Cl. CPC A61B 17/0206; A61B 2017/0237

(57) ABSTRACT

A mounting mechanism for use with surgical retractor rakes mounts to a fixed frame at an edge of a surgical site to that the rake can be conveniently configured into position with simple manual adjustments. A valve rake finger is arranged on the end of a shaft. An articulating joint having a movable plunger with a shaft carriage and a pressing mechanism selectably engages the plunger to lock the shaft in a desired position. A slider plate receives the articulating joint and attaches to a fixed frame.

### CROSS REFERENCE TO RELATED APPLICATIONS

This application claims priority to provisional application Ser. No. 62/372,334, filed on Aug. 9, 2016, which is incorporated herein by reference in its entirety.

### BACKGROUND OF THE INVENTION

The invention applies to heart surgery, especially heart valve repair or replacement. In the case of a damaged valve, open heart surgery is typically conducted to replace or repair the valve through the aorta. For this operation, operating room assistant personnel may use a valve rake to keep the valve round and stable for suturing.

Besides manually holding the valve rake, fixed mechanisms have been used to suspend a rake in a desired position. During cardiac surgery, a sternal retractor is typically mounted over the patient carrying retractor blades to separate overlying tissues to allow access to a surgical site. The frame of the sternal retractor has been used to support mechanical rakes, as in U.S. Pat. No. 5,772,583, for example.

To avoid complicated position adjustment mechanisms, it is also known to employ a flexible shaft for carrying the valve rakes. However, manipulation to obtain the desired position may require significant skill and may be inconvenient and time consuming. Furthermore, the size/thickness and length requirements for flexible structures that can also provide sufficient stability for the desired holding characteristics has also resulted in large mechanisms that consume needed space within the surgical area, especially since several valve rakes may be needed simultaneously.

### SUMMARY OF THE INVENTION

The invention uses a malleable shaft and a flexible joint. A surgeon or assistant can easily set a mandated position using just one locking dial. The mechanism provides fast, simple, and secure adjustment in a compact design that maintains available space for surgical access.

The mechanism provides degrees of freedom around three separate rotational axes and along one longitudinal axis, all simultaneously locked by one dial operation (e.g., a thumb screw). In addition, a malleable section can be provided at an intermediate location on the shaft to achieve additional fine adjustment of the rake position.

### BRIEF DESCRIPTION OF THE DRAWINGS

FIG. 1 is a top view of one example of a conventional sternal retractor system.

FIG. 2 is a perspective view of another example of a conventional sternal retractor system.

FIG. 3 is a perspective view of a retractor rake mounting system according to one preferred embodiment of the invention.

FIG. 4 is a perspective, exploded view of a mounting mechanism of the embodiment of FIG. 3.

FIG. 5 is a perspective view with portions of the mounting mechanism removed to show various degrees of freedom for adjusting the position of a retractor.

FIGS. 6 and 7 are perspective views of the mounting mechanism with various elements removed.

FIGS. 8 and 9 are cross-sectional views showing interaction between the pusher and the swing base of FIG. 4.

FIGS. 10-12 are perspective views showing interaction among the swing top, pusher, swing base, and plunger of FIG. 4.

FIG. 13 is a perspective view showing the end cap of FIG. 4 in greater detail.

FIG. 14 is a top view of the swing base with the plunger installed in a corresponding slot.

FIG. 15 is a bottom view of the swing top.

FIGS. 16 and 17 are perspective views showing the retractor rake in greater detail.

FIG. 18 is a top view of a sternal retractor system having retractor rakes mounted thereto using a mounting mechanism of the present invention.

### DETAILED DESCRIPTION OF PREFERRED EMBODIMENTS

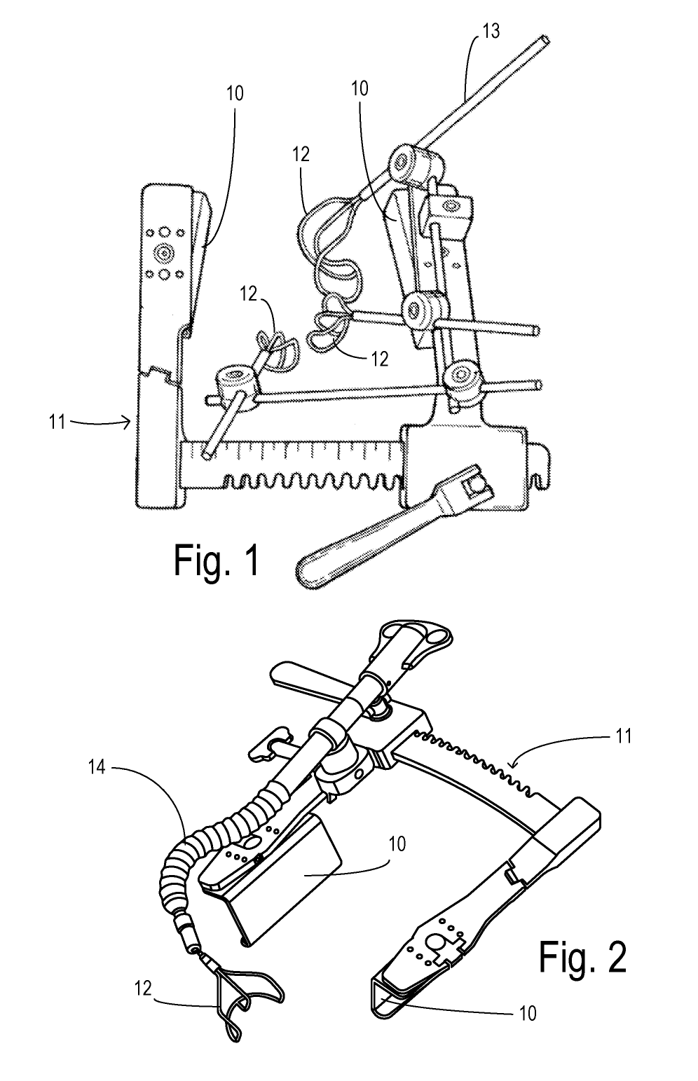

FIGS. 1 and 2 show prior art sternal retractors with spaced blades 10 carried by an adjustable frame 11. Valve rakes 12 are carried by rigid rods 13 or bendable rod 14, respectively. Each may have an adjustable suspension mechanism.

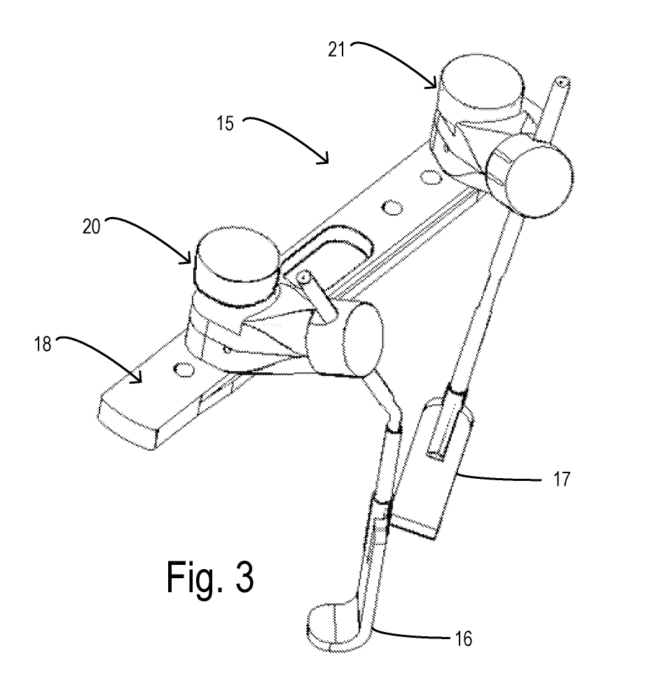

FIG. 3 shows a first embodiment of the invention wherein retractor rake fingers 16 and 17 are attached to a retractor slide 18 by mounting mechanisms 20 and 21, respectively, which are constructed as identical articulating joints. Slide 18 is configured to attach to a frame of a sternal retractor system or to other fixed structures depending on the particular surgical procedure for which it may be used. Mechanism 20 will be described in greater detail below.

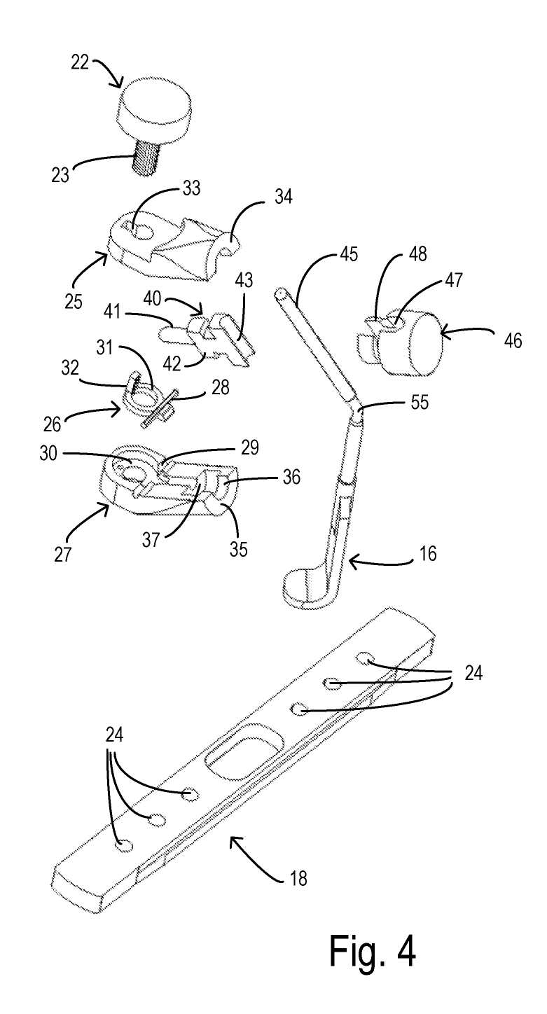

FIG. 4 shows an exploded view wherein mechanism 20 is comprised of a thumb screw 22 having a threaded shaft 23 which matches any of threaded receiving holes 24 in slider 18. Slider 18 can be fixed to the frame of the sternal retractor in any convenient manner known to the art. Threaded shaft 23 of thumb screw 22 passes through respective bores in an upper swing top 25, a pusher 26, and a lower swing base 27. Pusher 26 has a pivot pin 28 received in a hinge slot 29 in swing base 27. Base 27 has a socket 30 for receiving a ring portion 31 of pusher 26. A push bar 32 extends from pusher 26 to extend through a keyhole 33 in swing top 25. Swing top 25 and swing base 27 have matching collar extensions 34 and 35 defining an internal bore 36 and a cylindrical slot 37. A plunger member 40 has a shaft 41 received in bore 36, a guide bar 42 to be rotationally received in slot 37, and a carriage bar 43 for receiving a shaft 45 of rake 16. An end cap 46 has a diametric slot 47 for receiving a shaft 45 of rake 16 and carriage bar 43. A retention flange 48 projecting from side prongs of cap 46 is adapted to fit into slot 37 along with guide bar 42, and the entire structure can be locked in place by inserting thumbscrew shaft 23 into holes 24 and tightening thumbscrew 22.

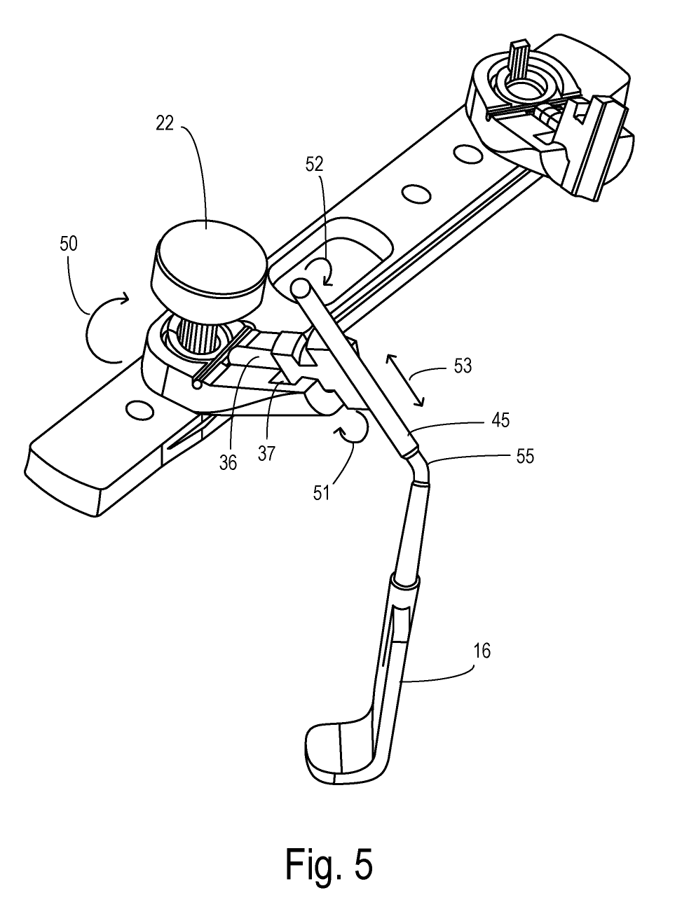

FIG. 5 shows mechanism 20 with the end cap and swing top removed. Before locking down the mechanism by tightening thumbscrew 22, valve rake 16 and shaft 45 can be easily moved according to several degrees of freedom including a rotational axis 50 around the axis of thumbscrew 22, a rotational axis 51 defined by bore 36 and slot 37, a rotational axis 52 around the central axis of shaft 45, and a longitudinal axis 53 along shaft 45. In addition, shaft 45 and valve rake 16 may be joined by a bendable shaft section 55 (e.g., made of a malleable material) for additional degrees of freedom.

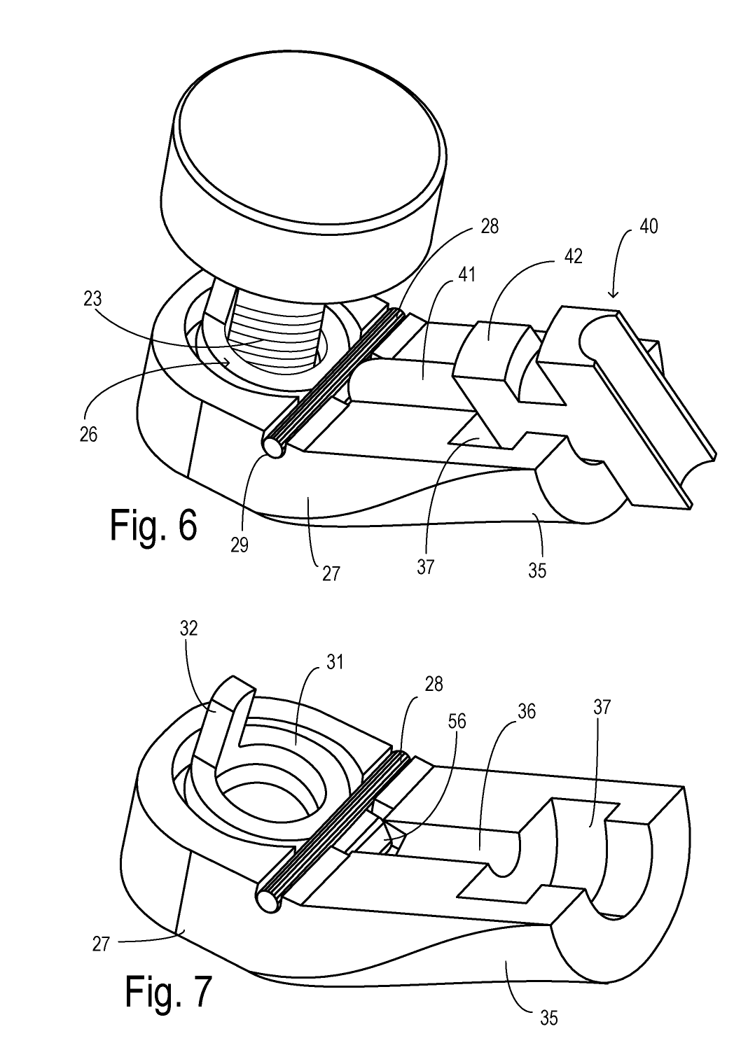

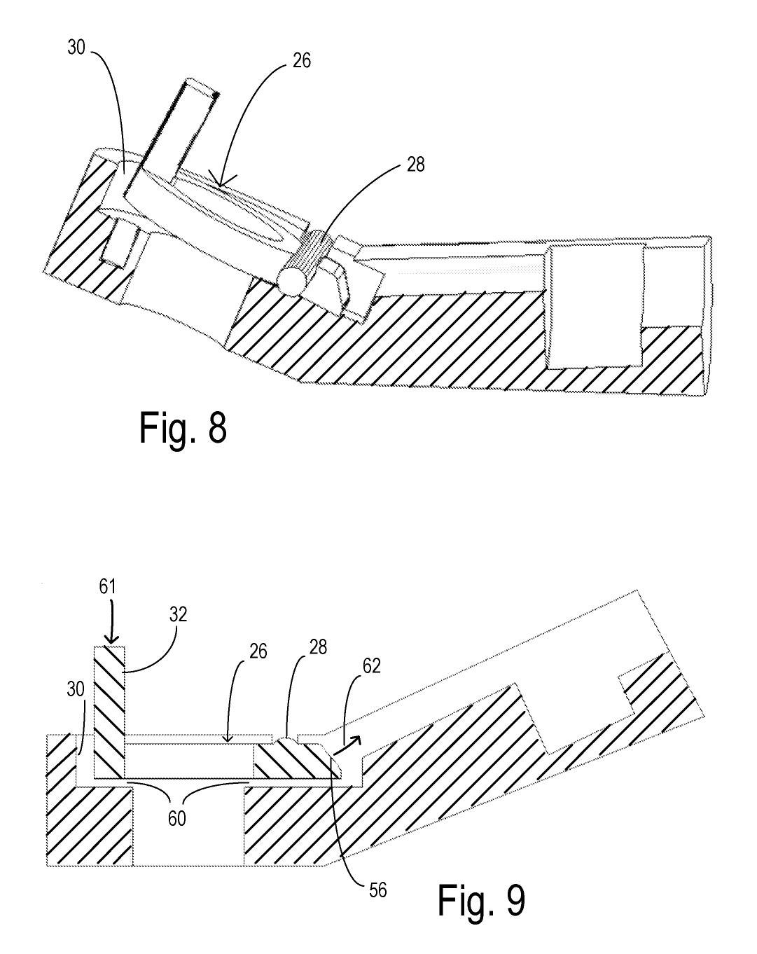

As shown in FIGS. 6 and 7, plunger member 40 is slidable along the direction of shaft 41 in bore 36 and slot 37 under control of a tappet 56 extending from ring portion 31 of pusher 26. As shown in FIGS. 8 and 9, pusher 26 is rotatable around pivot pin 28 by providing a clearance 60 between pusher 26 and the inner surface of socket 30. Thus, when the thumb screw is tightened, then a bottom surface of the head of the thumbscrew presses downward along a force line 61 against push bar 32 so that tappet 56 rotates upward along arrow 62 in order to press against the plunger member which forces the plunger upward into the end cap, which locks the shaft of the rake into place. In the tightened condition with pusher 26 forcing swing base 27 down against retractor slide 18 and with tappet 56 engaging plunger member 40 so that end cap 46 and rake shaft 45 become bound in place, all degrees of freedom are removed (except for the bendable section, if any, on the rake finger shaft).

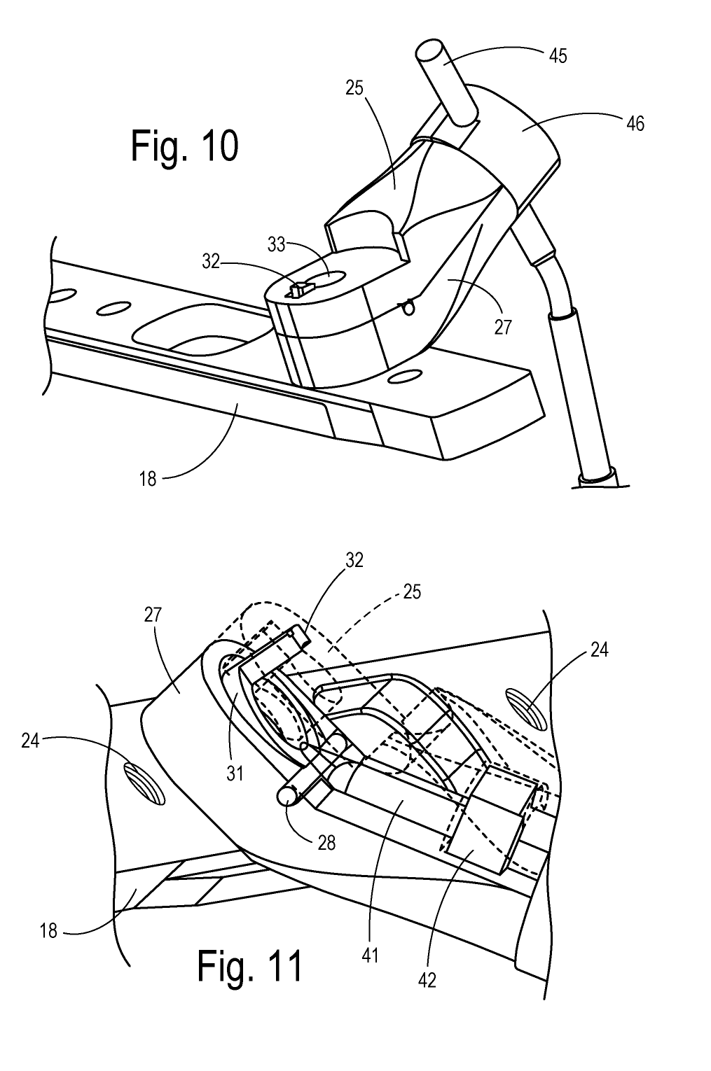

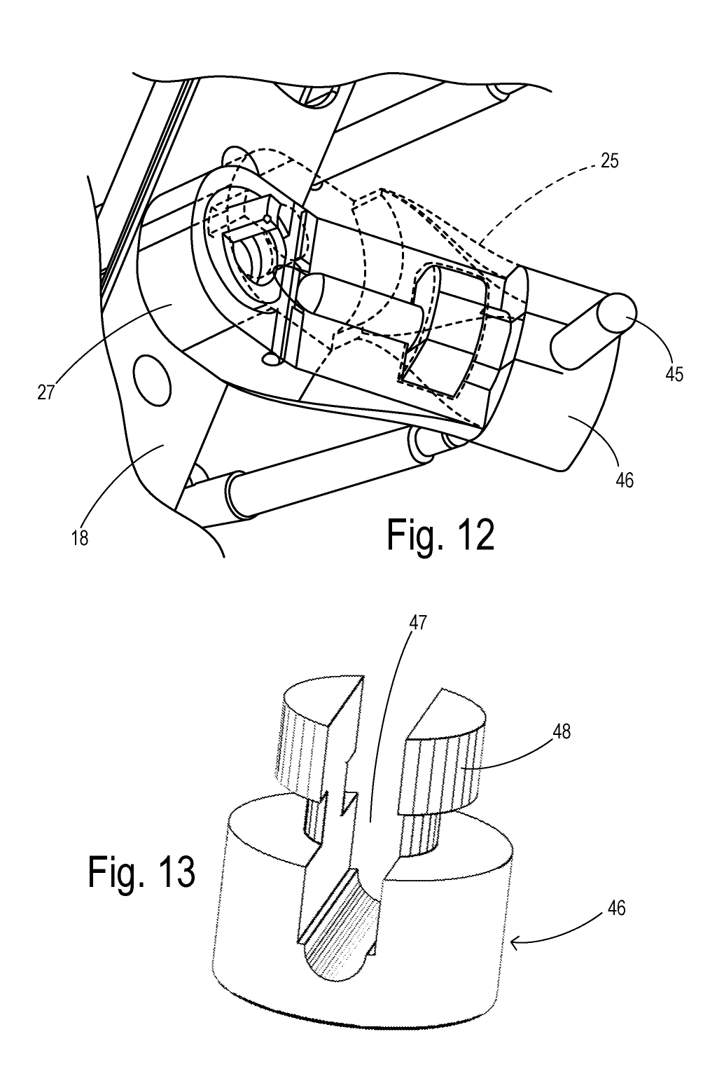

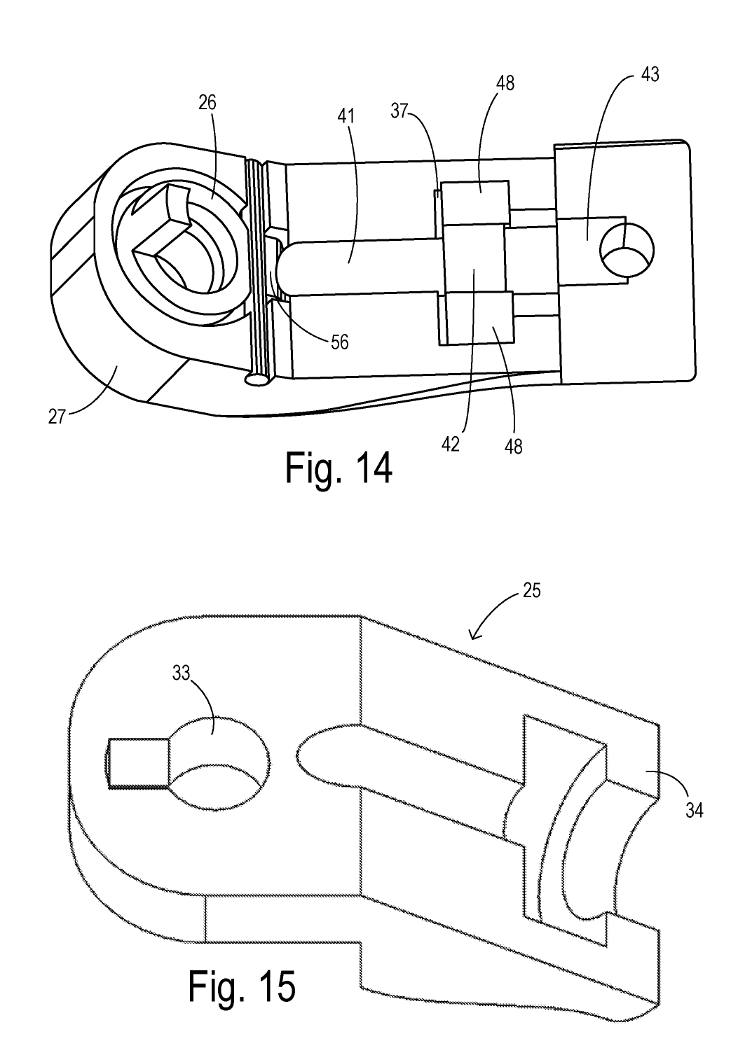

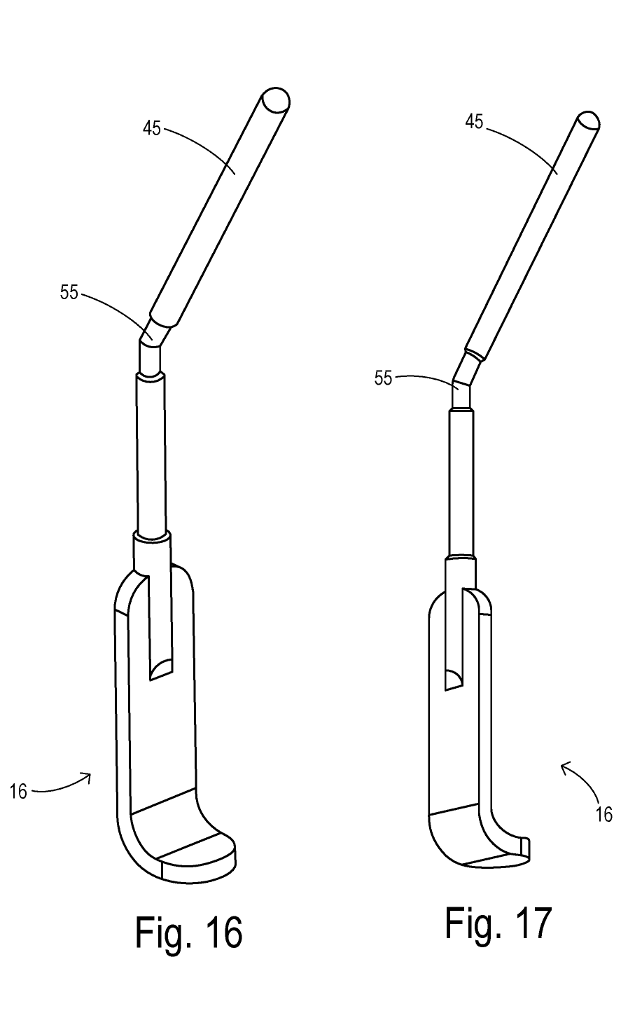

FIG. 10 shows lower swing base 27 and upper swing top 25 in place over retractor slide 18. Thumb screw 22 is removed to reveal push bar 32 extending out of keyhole 33 in order to interact with thumb screw 22. FIGS. 11, 12, and 14 show how rotation of ring portion 31 and push bar 32 around pivot pin 28 translates into longitudinal motion of shaft 41 within limits set by movement of guide bar 42 within slot 37. FIG. 14 in particular shows that the longitudinal width of guide bar 42 on plunger 40 is shorter than the corresponding longitudinal width of slot 37 in order to allow sliding movement along the bore.

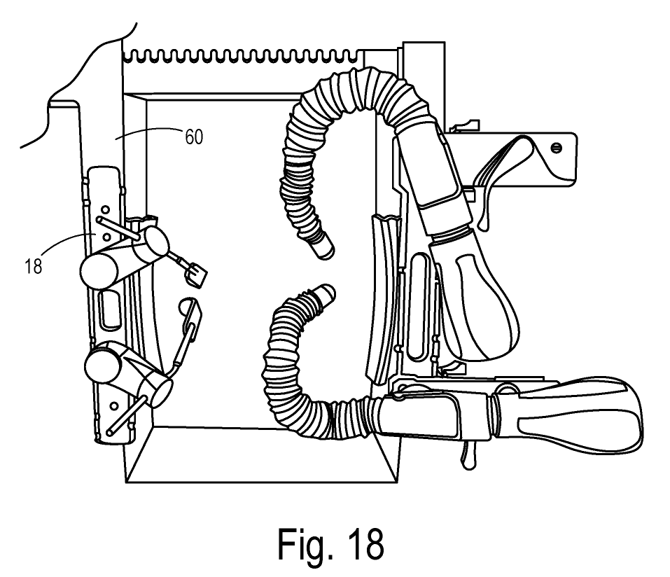

FIG. 18 shows a frame 60 receiving retractor slider 18. The space used by the mechanism of the present invention on the left side of the figure is significantly reduced as compared to a prior art flexible suspension for the valve rake on the right side of figure.

The invention can be used to hold retractor fingers to hold various tissues for many types of surgical operations. In the specific example of aortic valve repair or replacement, the invention may be used according to the following steps:
Step 1: Put sternal retractor on the chest after cutting a sternal incision.
Step 2: Expose heart and cut aorta. Expose valve through aorta.
Step 3: Set finger tool (rake) onto the retractor mounting mechanism.
Step 4: Loosen dial to release rake. Use freedom of movement of the rake to secure a valve body in a desired position for suturing. Degrees of freedom include:
   (A) Yaw axis (coaxial with dial),
   (B) Pitch (rotate portion on rake shaft holder),
   (C) Roll (rake shaft rotation), and
   (D) Longitudinal slide of rake shaft to adjust preferable arrangement.
Step 5: Tighten thumb screw to lock the positions of the mounting mechanism and rake.
Step 6: If necessary repeat step 4&5.

What is claimed is:

1. Apparatus comprising:
a valve rake finger on the end of a shaft;
an articulating joint having a movable plunger with a shaft carriage and a pressing mechanism for selectably engaging the plunger to lock the shaft in a desired position; and
a retractor plate for receiving the articulating joint.

2. The apparatus of claim 1 wherein the shaft includes a malleable section between the valve rake finger and the shaft carriage.

3. An articulating joint for securing a shaft of a retractor rake to a fixed support, comprising:
a swing base with a socket, a hinge slot, and a collar extension defining a bore with a cylindrical slot;
a pusher pivotably received in the socket, wherein the pusher has a pivot pin, a push bar, and a tappet, wherein the pivot pin is received in the hinge slot, and wherein the pivot pin is disposed between the push bar and the tappet;
a plunger member slidably disposed in the bore and having a guide bar disposed in the cylindrical slot, wherein the plunger member has a shaft engaged with the tappet at one end and has a carriage bar at the other end for engaging the shaft of the rake;
an end cap having a slot for receiving the carriage bar and the shaft of the rake, wherein the end cap has a retention flange disposed in the cylindrical slot; and
a thumb screw securing the swing base to the fixed support and selectably compressed against the push bar to selectably lock the articulating joint into a fixed arrangement.

4. The articulating joint of claim 3 further comprising a swing top held against the swing base, the pusher, the plunger member, and the end cap by the thumb screw.

5. The articulating joint of claim 4 wherein the thumb screw comprises a threaded shaft adapted to engage threads on the fixed support, and wherein the swing base, pusher, and swing top have aligned holes for passing the threaded shaft.

6. The articulating joint of claim 3 wherein the plunger member is rotatable within the bore.

7. The articulating joint of claim 3 wherein the tappet is comprised of a slanted surface.
